(19) United States
(12) Patent Application Publication
(10) Pub. No.: US 2001/0053927 A1
(43) Pub. Date: Dec. 20, 2001

(54) Warming table for veterinary surgical procedures

(76) Inventor: Chris Bradley, Baltimore, MD (US)

Correspondence Address: Richard C. Litman, LITMAN LAW OFFICES, LTD., P.O. Box 15035, Arlington, VA, 22215, US

(21) Appl. No.: 09/861,616

(22) Filed: May 22, 2001

Related U.S. Application Data
(60) Provisional application No. 60/208,569, filed on Jun. 2, 2000.

(51) Int. Cl. A61F 7/00

(52) U.S. Cl. 607/104

(57) ABSTRACT

A medical table system which respectively supports the weight and peripherally regulates the temperature of a body in contact therewith by convective heat transfer. The system includes at least one table element having at least one fluid inlet for supplying temperature and pressure regulated influent therethrough. At least one fluid outlet is also disposed within a portion of the support structure for supplying temperature and pressure regulated effluent therethrough. The table system includes wherein each fluid inlet and outlet are connected intermediate of a plurality of fluid sub channels which supply fluid flow in a flow path substantially orthogonal to the first and second channels. A plurality of table support manifolds provide structural reinforcement to the table, including a lifting support assembly which provides selective height adjustments to the medical table system to form selective geometric configurations.

### CROSS-REFERENCE TO RELATED APPLICATION

This application claims the benefit of U.S. Provisional Patent Application Ser. No. 60/208,569, filed Jun. 2, 2000.

### BACKGROUND OF THE INVENTION

1. Field of the Invention

The present invention relates generally to medical tables. More specifically, the invention is a temperature controlled medical table system for regulating the contact surface temperature between the table of the system and another body or plurality of bodies disposed thereon.

2. Description of Related Art

Various therapeutic mats have been devised as heating sources for operating tables, beds, etc. In the veterinarian practice, heating mats are widely used in surgical rooms, holding cages and the like. These conventional items provide heat to a body of a surgical specimen via electrical heating methods which are often difficult to use or deploy and manipulate during pre-operative and post-operative procedures. Some of the conventional aforementioned features are disclosed by patents issued to W. M. Short (U.S. Pat. No. 1,970,200), Hopper et al. (U.S. Pat. No. 5,251,347) Theilacker et al. (U.S. Pat. No. 5,138,138) and Cranston et al. (U.S. Pat. No. 5,324,911).

Some of the most notable problems related to the use of heating mats are mat deterioration, electrical injury or the potential for disease migration from one user to the next due to repeated use. While some conventional techniques have tried to avoid these problems by inventing devices which use fluid therapy instead of electrical heating methods, owners of these conventional systems have discovered that even these methods require the use of expensive equipment which is either difficult to maintain or has associated maintenance costs which proves infeasible or impractical to use. Also, these devices require or demands a certain level of physical prowess to move from one location to the next. In sum, these noted problems, are impractical and inefficient, particularly for a small veterinary or other practice. Further, these particular practical deficiencies are disclosed by patents issued Bell et al. (U.S. Pat. No. 5,411,541), Truglio (U.S. Pat. No. 5,433,741), Palmer (U.S. Pat. No. 5,713,834) and Augustine et al. (U.S. Pat. No. 5,800,480).

Other conventional devices commonly used in the relevant industry are identified by patents respectively granted and issued to D. 0. Haughey (U.S. Pat. No. 3,217,672), Anderegg (U.S. Pat. No. 3,771,782), Demand (U.S. Pat. No. 4,491,173), Walker (DE 2115392), Erwin (DE 2308214), Rene (FR 2435678), Bythell et al. (2,098482) and Marielle (EP0743055). Of particular note, these conventional devices disclose standard heating tables and hydromassage features which are consider to be of general relevance to the medical table system as herein described.

None of the above inventions and patents, taken either singly or in combination, is seen to describe the instant invention as claimed.

### SUMMARY OF THE INVENTION

The medical table system according to the invention has table elements which respectively support the weight and peripherally regulate the temperature of a body or surgical specimen in contact therewith by convective heat transfer. The system includes at least one table element having at least one fluid inlet for supplying temperature and pressure regulated influent therethrough. At least one fluid outlet channel or line is also disposed within a portion of the support structure of the medical table for supplying temperature and pressure regulated effluent therethrough.

A first and second fluid channel is disposed within a manifold support. Each of the channels is respectively made contiguous with and fluidly connected to at least one fluid inlet and outlet. The table system includes wherein each fluid inlet and outlet are connected intermediate of a plurality of fluid sub channels which supply fluid flow (i.e. gas, vapor or liquid) in a flow path which is conveyed substantially in orthogonal relation with respect to the first and second channels.

As a structural reinforcement measure, a plurality of table support manifolds are disposed on a bottom portion of the table element for uniformly distributing and supporting a predetermined weight capacity of a body or surgical specimen mounted thereon. A lifting support assembly is provided for selectively rotating and positioning the medical table system. Easily removable mechanical fasteners are used to secure the system in a fixed V-shaped or alternative configuration.

Accordingly, it is a principal object of the invention to provide a medical table system for veterinary medical procedures.

It is another object of the invention to provide a medical table system which is an expandable medical table platform.

It is a further object of the invention to provide a medical table system which regulates the peripheral temperature of a body or specimen by convective heat transfer.

Still another object of the invention is to provide a medical table system which is adjustable for a variety of different heights and geometric configurations for supporting a surgical specimen (i.e. animal, human, etc.).

Further still, it is an object of the invention to provide a medical table system which is made of a material which is impervious to stress related fractures and which is made of a light-weight polymeric or composite polymeric material.

It is an object of the invention to provide improved elements and arrangements thereof in the medical table apparatus for the purposes described which is inexpensive, dependable and fully effective in accomplishing its intended purposes.

These and other objects of the present invention will become readily apparent upon further review of the following specification and drawings.

### BRIEF DESCRIPTION OF THE DRAWINGS

FIG. 1 is an environmental, perspective view of a medical table system for veterinary surgical procedures according to the present invention.

FIG. 2 is an exploded perspective view of the medical table according to the first embodiment.

FIG. 3 is a sectional perspective view of FIG. 2, taken along line 3-3, illustrating fluid flow channels according to the first embodiment of the invention.

FIG. 4 is a perspective view of the medical table system as a matrix of interconnected elements, illustrating a system of linked fluid connections according to the first embodiment.

FIG. 5 is an exploded perspective view of the medical table system according to a second embodiment.

Similar reference characters denote corresponding features consistently throughout the attached drawings.

### DETAILED DESCRIPTION OF THE PREFERRED EMBODIMENTS

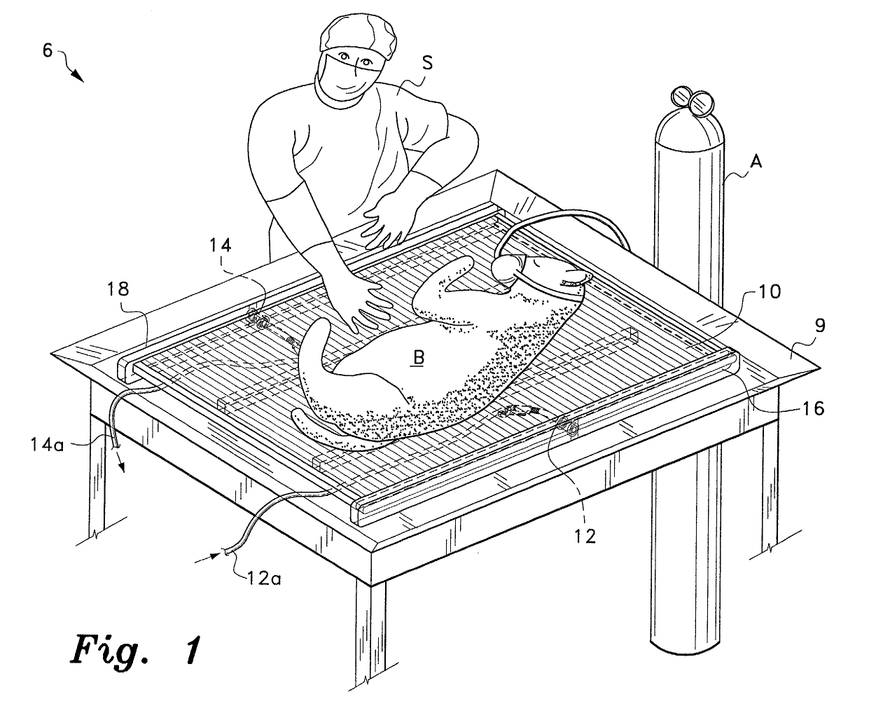

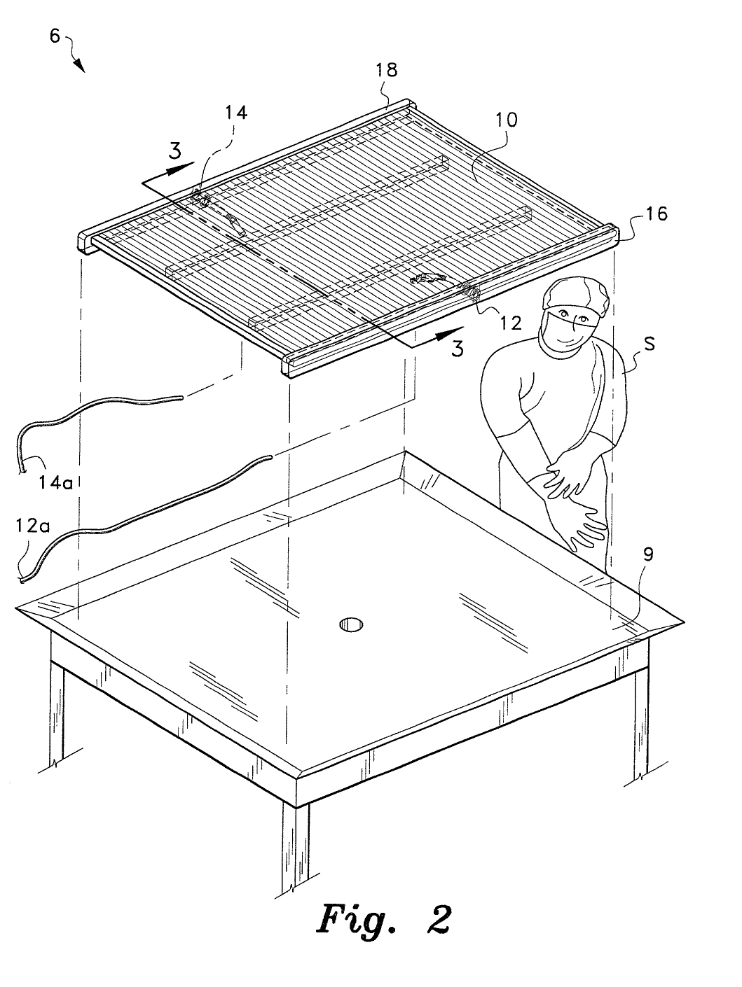

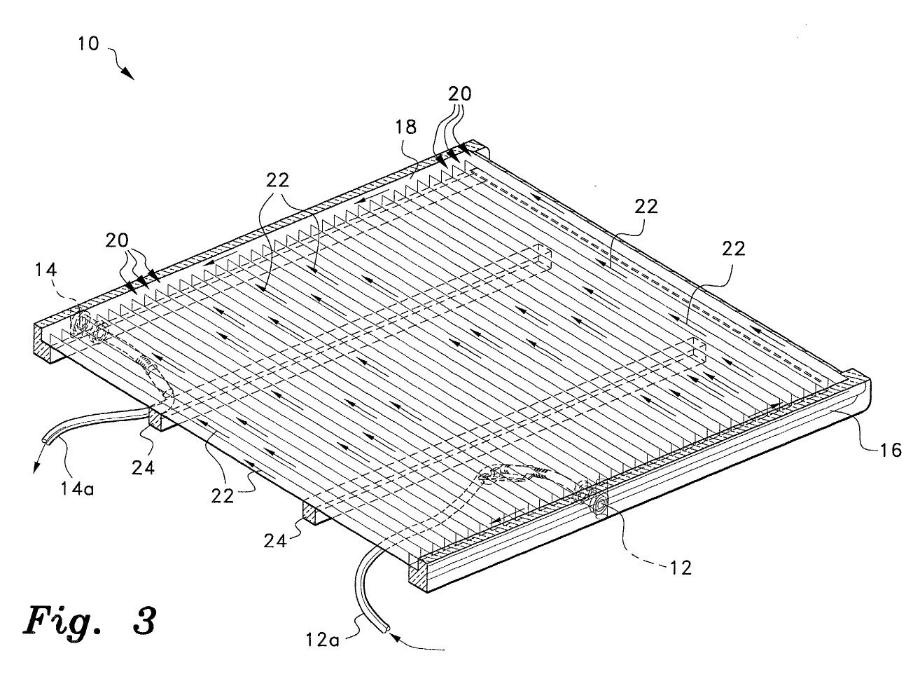

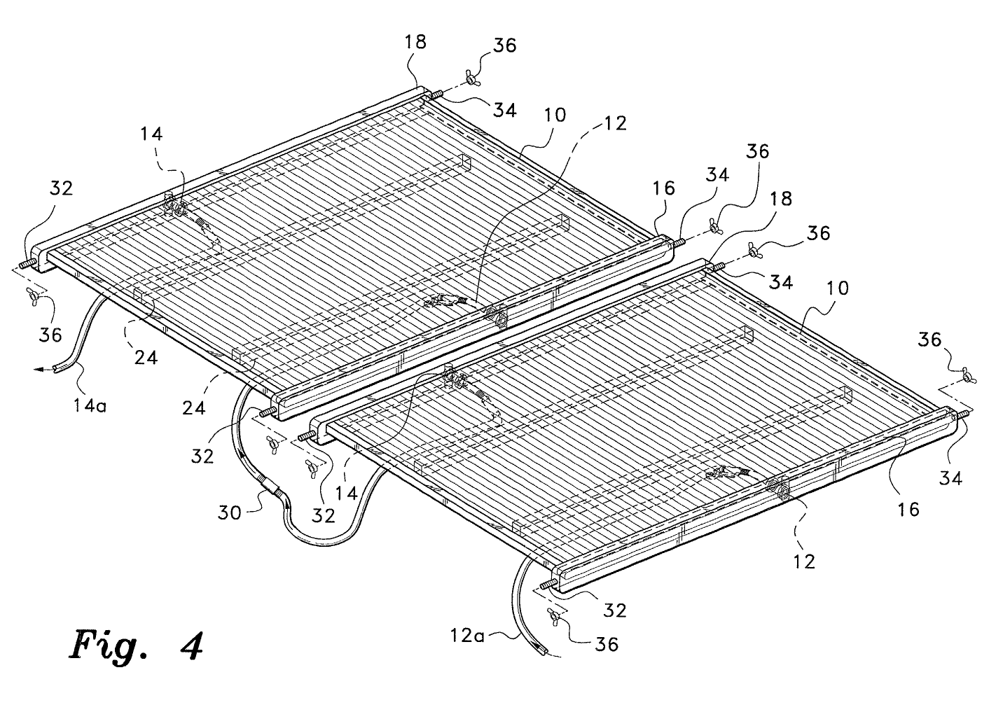

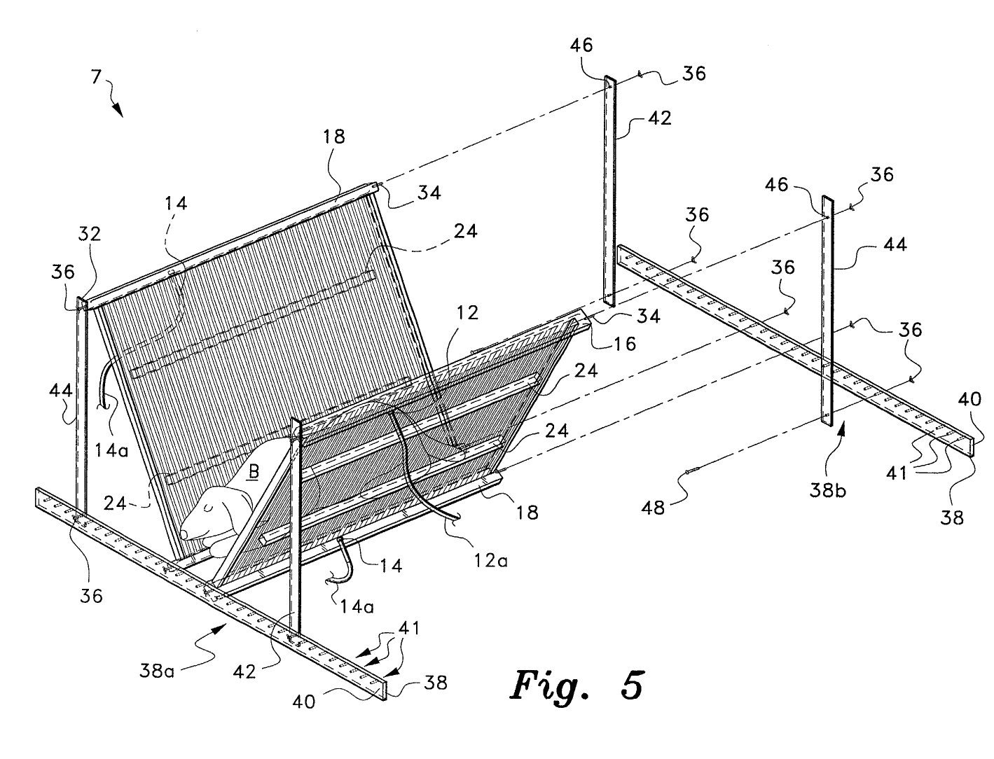

The present invention is directed to a medical table system for respectively supporting and peripherally regulating the temperature of a body or surgical specimen B (i.e. animal, human, etc.) supported in contact therewith through convective heat transfer. The preferred embodiments of the present invention are depicted in FIGS. 1-3, according to a first embodiment 6. With respect to other illustrative features, FIGS. 4 and 5 shows the invention according to a second embodiment 7.

As best seen in FIGS. 1-3, a medical professional S is shown utilizing the medical table 6 for implementing a medical procedure which includes the supply of anesthesia A to a specimen B. The medical table 6 is supported by an auxiliary support means 9, such as a medical table or the like and/or a recovery cage or similar object respectively used in pre-operative and post-operative procedures. The table 6 comprises at least one table element 10 having at least one fluid inlet 12 for supplying temperature and pressure regulated influent 12a therethrough, and at least one fluid outlet 14 for supplying temperature and pressure regulated effluent 14a therethrough, as respectively indicated by the direction arrows made adjacent thereto.

The table 10 further comprises a first fluid channel 16 and a second fluid channel 18. The first fluid channel 16 is preferably made contiguous with and fluidly connected to at least one fluid inlet 12. Similarly, the second fluid channel 18 is made contiguous with and fluidly connected to at least one fluid outlet 14. Fluid inlet 12 and outlet 14 are also fluidly connected via an intermediate plurality of sub channels 20. The sub channels 20 are integrally formed within the medical table as substantially rectilinear channels which provide uniform fluid flow as indicated by the arrows 22. The medical table element 10 is preferably made of a material available under the trademark LEXAN.

Other materials and manufacturing processes can be used to produce the product material, (eg. composite plastics, injection molding, etc.) so long as the materials have similar properties indicative of LEXAN, and are used within the scope of the invention as herein disclosed. The plurality of sub channels 20 are arranged substantially orthogonal to respective first and second channels 16,18, and supplies fluid flow therethrough in a flow path made orthogonal to channels 16 and 18, respectively. A plurality of table support manifolds 24 are disposed on an under-surface portion of the medical table element 10 for uniformly distributing and supporting the weight of the body B throughout the surface area of medical table element 10.

As diagrammatically illustrated in FIG. 4, the medical table element 10 is shown in a matrix arrangement, wherein at least one medical table element 10 further comprises a means 30 for linking at least one effluent channel 14a of one the table elements 10 to an inlet of another one of the medical tables 10. This particular arrangement supplies a continuous flow of fluid in either liquid, gas, or vapor phase therethrough. In an expanded arrangement, the linked tables can be configured to form a larger matrix of medical tables 10 for providing an extended temperature regulated surface area, depending on the number of bodies or the respective surface area needed for a single body or a plurality thereof. It should be noted that the plurality of manifolds 24 are preferably substantially rectangular in shape and are uniformly disposed on the under-surface portion of each medical table element 10.

Within the expanded matrix arrangement, the manifolds 24 are disposed in spaced arrangement from each other and are disposed parallel to the first and second channels 16,18. This particular arrangement provides maximum support with minimum material requirements for each manifold 24. In addition, the medical table system 6 includes wherein the first and second channels 16,18 of each table element 10 of the matrix or system 6, further comprises a respective first 32 and second 34 extension post on respective ends of the channels 16 and 18. Accordingly, the respective first 32 and second 34 extension posts are disposed on opposing ends of each channel 16 and 18, respectively. These posts include mating mechanical fasteners 36 or butterfly screws for securing a medical table lifting support assembly 38 thereto.

As diagrammatically illustrated in FIG. 5, the support assembly 38 is shown in more detail according to the second embodiment 7. The medical table system 7 is shown as a matrix of medical table elements 10 which are mounted to the support assembly 38 to form a substantially V-shaped configuration. Fluid which flows within the system 7 is similarly transported as recited above via at least one inlet 12 and outlet 14. The method of pumping fluid therethrough is well known in the relevant art and is not discussed in detail. However, any suitable method of pumping temperature controlled (warm or cool) and pressure regulated fluid therethrough can be used, so long as it is performed within the scope and intent of the invention as herein disclosed.

The medical table support assembly 38 is designed primarily to selectively rotate, lift and secure each table element 10 of the system 7 to a predetermined height in a secured position via mechanical fasteners 36. Operatively described, the medical table system 7 is rotatably adjusted via a first 38a and second 38b set of table supports as shown in FIG. 5. Each set of supports 38a, 38b comprises a base bar 40 having a plurality of uniformly spaced apertures 41 disposed therein, and a first 42 and second 44 vertical support bar or post. Each vertical post 42,44 comprises a first aperture 46 disposed on one end of each vertical post 42,44 and an extension post or threaded mechanical fastener 48 disposed on an opposite or opposing end of each post, respectively. The medical support assembly 38 operatively attaches to the extension posts 32,34 disposed on each table element 10, wherein the base bars 40 are secured to respective posts 42,44 as male and female connections.

The extension posts 32,34 on the respective ends of the channels 16,18 of each table element 10 further include a mating mechanical fastener 36 for selectively securing the medical table support assembly 38 in a fixed and mechanically secured configuration. Other advantages of the medical table systems 6 and 7 include, wherein the tables are made of a material which is not susceptible to the accumulation of residual bacteria from pre-operative and/or post-operative procedures. Unlike the currently available heating mats, the medical table system herein disclosed is practically indestructible and reusable. It is also noted, that the regulated fluid can be supplied in either liquid, gas or vapor phases as determined by one having ordinary skill in the relevant art.

It is to be understood that the present invention is not limited to the sole embodiments described above, but encompasses any and all embodiments within the scope of the following claims.

I claim:

1. A medical table element for peripherally regulating a body temperature in contact therewith by convective heat transfer, comprising, in combination with a support means for pre-operative and post operative medical procedures:
at least one table element having at least one fluid inlet for supplying temperature and pressure regulated influent therethrough and at least one fluid outlet for supplying temperature and pressure regulated effluent therethrough, said table further comprises a first fluid channel and a second fluid channel, said first fluid channel being contiguous with and fluidly connected to said at least one fluid inlet and said second fluid channel being contiguous with and fluidly connected to said at least one fluid outlet; said at least one fluid inlet and fluid outlet being fluidly connected via an intermediate plurality of sub channels, said sub channels being arranged to supply fluid flow in a flow path substantially orthogonal to said first and second channels first; and
a plurality of table support manifolds for uniformly distributing and supporting the weight of the body throughout out the at least one medical table.

2. The medical table according to claim 1, wherein: said at least one medical table further comprises a means for linking the at least one effluent channel to an inlet of another of said at least one medical tables for supplying continuous fluid therethrough, the linked tables forming a matrix of medical tables for providing an extended temperature regulated surface area for at least one body.

3. The medical table according to claim 1, wherein said plurality of manifolds being substantially rectangular in shape and ar uniformly disposed on a surface of said at least one medical table in spaced arrangement from each one of the plurality of manifolds and parallel to said first and second channels.

4. A medical table system for peripherally regulating a body temperature in contact therewith by convective heat transfer, comprising:
at least one table having at least one fluid inlet for supplying temperature and pressure regulated influent therethrough and at least one fluid outlet for supplying temperature and pressure regulated effluent therethrough, said table further comprises a first fluid channel and a second fluid channel, said first fluid channel being contiguous with and fluidly connected to said at least one fluid inlet and said second fluid channel being contiguous with and fluidly connected to said at least one fluid outlet; said at least one fluid inlet and fluid outlet being fluidly connected via an intermediate plurality of sub channels, said sub channels being arranged to supply fluid flow in a flow path substantially orthogonal to said first and second channels first;
a plurality of table support manifolds for uniformly distributing and supporting the weight of the body throughout out the at least one medical table;
a means for linking the at least one effluent channel to an inlet of another of said at least one medical tables for supplying continuous fluid therethrough, the linked tables forming a matrix of medical tables for providing an extended temperature regulated surface area for at least one body; and
a medical table support assembly for selectively rotating, lifting and securing each table in the matrix to a predetermined height and secured position within said support assembly.

5. The medical table system according to claim 4, wherein said first and second channels of each table of said matrix further comprise a respective first and second extension post, said respective first and second posts being disposed on opposing ends of said respective first and second channels for supporting the medical table lifting support assembly.

6. The medical table system according to claim 4, wherein said support assembly is an adjustable assembly comprising a first and second set of supports, each of said sets comprising a base bar with a plurality of uniformly spaced apertures and a first and second vertical support bar, said first and second vertical posts comprising a first aperture disposed on one end of said first and second vertical posts and an extension post disposed on an opposite end of said first and second post as male and female connectors for connecting to said base bar and said extension posts of each respective table in said matrix.

7. The medical table system according to claim 6, wherein said first and second extension posts of said first and second channels further include a mating mechanical fastener for selectively securing the medical table support assembly in a fixed and mechanically secured configuration.

8. The medical table system according to claim 4, wherein system is made of a light-weight and durable plastic material.

9. A medical table system for peripherally regulating a body temperature in contact therewith by convective heat transfer, comprising, in combination with an apparatus for supplying anesthesia to said body:
at least one table having at least one fluid inlet for supplying temperature and pressure regulated influent therethrough and at least one fluid outlet for supplying temperature and pressure regulated effluent therethrough, said table further comprises a first fluid channel and a second fluid channel, said first fluid channel being contiguous with and fluidly connected to said at least one fluid inlet and said second fluid channel being contiguous with and fluidly connected to said at least one fluid outlet; said at least one fluid inlet and fluid outlet being fluidly connected via an intermediate plurality of sub channels, said sub channels being arranged to supply fluid flow in a flow path substantially orthogonal to said first and second channels first;
a plurality of table support manifolds for uniformly distributing and supporting the weight of the body throughout out the at least one medical table;
a means for linking the at least one effluent channel to an inlet of another of said at least one medical tables for supplying continuous fluid therethrough, the linked tables forming a matrix of medical tables for providing an extended temperature regulated surface area for at least one body; and
a medical table support assembly for selectively rotating, lifting and securing each table in the matrix to a predetermined height and secured position within said support assembly.

10. The medical table system according to claim 9, wherein said first and second channels of each table of said matrix further comprise a respective first and second extension post, said respective first and second posts being disposed on opposing ends of said respective first and second channels for supporting the medical table lifting support assembly.

11. The medical table system according to claim 9, wherein said support assembly is an adjustable assembly comprising a first and second set of supports, each of said sets comprising a base bar with a plurality of uniformly spaced apertures and a first and second vertical support bar, said first and second vertical posts comprising a first aperture disposed on one end of said first and second vertical posts and an extension post disposed on an opposite end of said first and second post as male and female connectors for connecting to said base bar and said extension posts of each respective table in said matrix.

12. The medical table system according to claim 11, wherein said first and second extension posts of said first and second channels further include a mating mechanical fastener for selectively securing the medical table support assembly in a fixed and mechanically secured configuration.

13. The medical table system according to claim 9, wherein said system is made of a light-weight and durable plastic material.
